(19) United States
(12) Patent Application Publication
(10) Pub. No.: US 2011/0142962 A1
(43) Pub. Date: Jun. 16, 2011

(54) Oral Rehydration Solutions Comprising Dextrose

(76) Inventors: Steven T. Luebbers, Columbus, OH (US); Julie J. Chio, Hilliard, OH (US); Pedro A. Alarcon, Gurnee, IL (US)

(21) Appl. No.: 12/950,991

(22) Filed: Nov. 19, 2010

Related U.S. Application Data
(60) Provisional application No. 61/285,630, filed on Dec. 11, 2009.

(51) Int. Cl. A61K 33/14; A61P 1/00; A61P 43/00

(52) U.S. Cl. 424/680

(57) ABSTRACT

The present disclosure is directed to oral rehydration solutions, and the use of these solutions to prevent dehydration due to fever. The oral rehydration solutions of the present disclosure may be oral rehydration solutions comprising about 12 mEq/L to about 18 mEq/L of sodium, from about 11 g/L to about 60 g/L of dextrose, a zinc source, and less than about 25 mEq/L of citrates. The pH of the oral rehydration solutions can be controlled by adjusting the amount of dextrose present in the oral rehydration solutions, allowing for formulation of oral rehydration solutions comprising lower levels of citrates.

This application claims the benefit of U.S. Provisional Application No. 61/285,630 filed Dec. 11, 2009

### BACKGROUND OF THE DISCLOSURE

The present disclosure is directed to oral rehydration solutions, and the use of the oral rehydration solutions to prevent dehydration resulting from fever. The oral rehydration solutions of the present disclosure may comprise from about 12 mEq/L to about 18 mEq/L of sodium, from about 5 g/L to about 90 g/L of dextrose, a zinc source, and less than about 25 mEq/L of citrates. The pH of the oral rehydration solutions can be controlled by adjusting the amount of dextrose present in the oral rehydration solutions, allowing for formulation of oral rehydration solutions comprising lower levels of citrates.

Fruit juices historically have been popular beverages for adult and child consumption. In addition to their palatability, fruit juices are considered to have nutritional value due to their content of vitamins, minerals, antioxidants and other components. One drawback to fruit juices, however, is their high content of sugar and calories. The high sugar content of fruit juices also has caused health professionals to discourage their use in the maintenance of hydration or in oral rehydration therapy (ORT).

ORT typically involves the administration of an oral rehydration solution (ORS) containing, at a minimum, glucose and sodium in water. An ORS provides rapid, effective hydration because sodium ion absorption in the intestines causes water molecules associated with the sodium ion to also be absorbed. This sodium absorption is activated by glucose. Specifically, every glucose molecule that crosses the intestinal epithelium brings a sodium ion with it, raising the concentration of ions in the blood stream and pulling water out of the gut. Sodium absorption improves as the glucose concentration of the oral fluid is increased up to about 2.5% w/w. At higher concentrations, the glucose can no longer be efficiently absorbed, leading to a net reduction in sodium and water absorption. In fact, higher concentrations of glucose increase the osmotic load in the gut, which pulls water out of the blood stream. This leads to a net loss of fluids and electrolytes, further exacerbating dehydration.

An ORS can thus be used to correct the fluid and electrolyte losses associated with acute infectious diarrhea and/or vomiting, to treat hyponatremia or hypohydration due to exercise, changes in altitude, or fever, and to maintain a healthy level of hydration. In fact, the use of ORT has significantly decreased the mortality rate associated with diarrhea, particularly in developing countries.

The World Health Organization (WHO) has recommended two ORS formulas. The initial formula has a glucose concentration of 111 mEq/L, a sodium concentration of 90 mEq/L, a potassium concentration of 20 mEq/L, a chloride concentration of 80 mEq/L, and a base concentration of 30 mEq/L. A more recent formula has a glucose concentration of 75 mEq/L, and a sodium concentration of 75 mEq/L.

A number of beverages are also available in the United States that are marketed as providing hydration. These beverages include Pedialyte® and Rehydralyte® (Abbott Laboratories; Abbott Park, Ill.); Enfalyte® (Mead Johnson & Company; Evansville, Ind.); CeraLyte® (Cera Products, Inc., Columbia, Md.); and Liquilytes® (Gerber Products Company; Parsippany, N.J.).

Currently available oral rehydration solutions typically contain relatively high amounts of citrates, i.e., about 30 mEq/L to about 40 mEq/L, or even more. The predominant source of citrates in oral rehydration solutions is citric acid, which is often added to an ORS to adjust the pH of the ORS to a desired level. However, high levels of citrates in an ORS may have certain undesirable effects in some patients. For instance, administration of ORS with high levels of citrate to children without diarrhea and metabolic acidosis may produce negative effects on the acid-base balance of these patients. It would thus be desirable to provide an ORS with a desired pH that has a reduced total citrate content.

It has now been unexpectedly discovered that the pH of an ORS can be controlled and adjusted by adjusting the amount of dextrose present in the ORS, and subjecting the ORS to heat sterilization, such as retort sterilization. By controlling the pH in the ORS with dextrose, the ORS can be prepared with the desired pH utilizing a lower amount of citrates than would otherwise be required to achieve the same pH. It has also been unexpectedly discovered that a greater reduction in the pH of the ORS is achieved using lower dextrose concentrations as compared to higher dextrose concentrations.

### SUMMARY OF THE DISCLOSURE

The present disclosure is directed to oral rehydration solutions, and the use of oral rehydration solutions to prevent dehydration due to fever. In one aspect, the oral rehydration solution comprises about 12 mEq/L to about 18 mEq/L of sodium, about 5 g/L to about 90 g/L of dextrose, a zinc source, and less than about 25 mEq/L of citrates, wherein the oral rehydration solution has been heat sterilized.

The present disclosure is further directed to a method of making an oral rehydration solution. The method comprises combining suitable amounts of water, dextrose, a sodium source, and a zinc source to form an oral rehydration solution comprising about 12 mEq/L to about 18 mEq/L of sodium, about 11 g/L to about 60 g/L of dextrose, the zinc source, and less than about 25 mEq/L of citrates; and heat sterilizing the oral rehydration solution.

The present disclosure is further directed to a method of preventing dehydration. The method comprises preparing an oral rehydration solution comprising about 12 mEq/L to about 18 mEq/L of sodium, about 11 g/L to about 60 g/L of dextrose, a zinc source, and less than about 25 mEq/L of citrates, wherein the oral rehydration solution has been heat sterilized; and orally administering the sterilized oral rehydration solution to an individual at risk of developing dehydration.

It has been unexpectedly discovered that the pH of an oral rehydration solution comprising about 12 mEq/L to about 18 mEq/L can be controlled by adjusting the amount of dextrose present in the oral rehydration solution, and subjecting the ORS to heat sterilization, such as retort sterilization. Specifically, the pH of an ORS including dextrose is lower following sterilization than the pH of the ORS prior to sterilization. It has further been discovered that the significance of the pH drop following sterilization depends on the amount of dextrose in the ORS, and that lower amounts of dextrose actually produce a greater decline in pH. By adjusting the amount of dextrose present in the ORS prior to sterilization, oral rehydration solutions having a desired pH and comprising lower levels of citrates can be formulated.

### BRIEF DESCRIPTION OF THE DRAWINGS

FIG. 1 is a graph showing the change in pH (A) following retort sterilization for oral rehydration solutions comprising 15 mEq/L of sodium and varying amounts of dextrose, as well as the linear regression line for these results, as discussed in Example 7.

### DETAILED DESCRIPTION OF THE DISCLOSURE

The present disclosure is directed to oral rehydration solutions comprising sodium, dextrose, and a zinc source, and the use of the oral rehydration solutions for the prevention of dehydration due to fever and/or other medical conditions not associated with diarrhea and vomiting. Methods for preparing an oral rehydration solution, including methods for controlling the pH of an oral rehydration solution are also disclosed. These and other essential or optional elements or limitations of the oral rehydration solutions and methods of the present disclosure are described in detail hereafter.

The term “infant” as used herein, unless otherwise specified, refers to children not more than about one year of age, and includes infants from 0 to about 4 months of age, infants from about 4 to about 8 months of age, infants from about 8 to about 12 months of age, low birth weight infants at less than 2,500 grams at birth, and preterm infants born at less than about 37 weeks gestational age, typically from about 26 weeks to about 34 weeks gestational age. The term “child” or “children” as used herein refers to children not more than 12 years of age, and includes children from about 12 months to about 12 years of age. The term “adult” as used herein refers to adults and children about 12 years and older.

One “milliequivalent” (mEq) refers to the number of ions in solution as determined by their concentration in a given volume. This measure is expressed as the number of milliequivalents per liter (mEq/L). Milliequivalents may be converted to milligrams by multiplying mEq by the atomic weight of the mineral and then dividing that number by the valence of the mineral.

Any reference to a numerical range in this application should be considered as being modified by the adjective “about”.

All percentages, parts and ratios as used herein, are by weight of the total solution, unless otherwise specified. All such weights as they pertain to listed ingredients are based on the active level and, therefore, do not include solvents or by-products that may be included in commercially available materials, unless otherwise specified.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

Any reference in the specification or claims to a quantity of an electrolyte should be construed as referring to the final concentration of the electrolyte in the ORS. Tap water often contains residual sodium, chlorine, etc. A value of 15 mEq of sodium, in this application, thus means that the total sodium present in the ORS equals 15 mEq, taking into account both added sodium as well as the sodium present in the water used to manufacture the ORS. This holds true for all electrolytes, including the mineral zinc.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The oral rehydration solutions of the present disclosure may also be substantially free of any optional or selected essential ingredient or feature described herein, provided that the remaining solution still contains all of the required ingredients or features as described herein. In this context, and unless otherwise specified, the term “substantially free” means that the selected solution contains less than a functional amount of the optional ingredient, typically less than 0.1% by weight, and also including zero percent by weight of such optional or selected essential ingredient.

The oral rehydration solutions and corresponding manufacturing methods of the present disclosure can comprise, consist of, or consist essentially of the essential elements and limitations of the disclosure as described herein, as well as any additional or optional ingredients, components, or limitations described herein or otherwise useful in oral rehydration applications.

### Oral Rehydration Solution Embodiments

### Dextrose

The oral rehydration solutions of the present disclosure comprise dextrose. The dextrose may be included in the ORS of the present disclosure in an amount of from about 5 g/L to about 90 g/L, including from about 11 g/L to about 60 g/L, or from about 11 g/L to about 36 g/L, or from about 11 g/L to about 24 g/L, or from about 24 g/L to about 60 g/L.

Including dextrose in the ORS in the amounts set forth herein allows the ORS to be formulated to have a desirable pH, for example, a pH of from about 3.0 to about 5.5, while using lower amounts of citric acid than would otherwise be required to achieve the desired pH. As discussed above, the pH of an ORS is often controlled by adding citric acid to the ORS, for example, in amounts of from about 0.5 g/L to about 2 g/L. However, including citric acid in an ORS in these amounts may result in undesirably high levels of citrates in the ORS. For example, a typical ORS includes citrates in amounts of from about 10 mEq/L to about 40 mEq/L, and more typically in amounts of from about 30 mEq/L to about 40 mEq/L. While an ORS containing citrates at these levels can stimulate intestinal absorption of sodium and chloride and can satisfactorily correct the metabolic acidosis in acute diarrhea associated with severe dehydration, if administered to children without diarrhea and metabolic acidosis it may produce negative effects on the acid-base balance of these patients.

It has now surprisingly been discovered that the pH of an ORS can be controlled by adjusting the amount of dextrose present in the ORS, and subjecting the ORS to a heat sterilization process, such as a retort sterilization, aseptic sterilization, or hot fill process. Specifically, in one embodiment of the present disclosure, an ORS including dextrose has a lower pH following retort sterilization, with the significance of the pH drop following retort sterilization depending on the amount of dextrose in the ORS.

The difference in the pH of the ORS prior to sterilization and the pH of the ORS following sterilization for oral rehydration solutions comprising 15 mEq/L of sodium and from about 5 g/L to about 90 g/L of dextrose ranges from about 0.11 to about 0.23. Unexpectedly, it has been discovered that larger pH drops following sterilization do not correlate with increasing levels of dextrose. Rather, the difference between pre- and post-sterilization pH is greater when lower levels of dextrose are included in the ORS. Specifically, dextrose levels of from about 11 g/L to about 36 g/L (pH drop of from about 0.18 to about 0.23), and more particularly from about II g/L to about 24 g/L (pH drop of from about 0.21 to about 0.23) result in the most significant pH drop following sterilization of an ORS comprising 15 mEq/L of sodium.

Because the inclusion of from about 5 g/L to about 90 g/L of dextrose in oral rehydration solutions comprising about 15 mEq/L of sodium results in a drop in pH following sterilization, reduced amounts of citric acid can be included in the oral rehydration solutions of the present disclosure, as compared to what would otherwise be required to achieve the same pH level. For example, the oral rehydration solutions of the present disclosure will preferably comprise only from about 0.3 g/L to about 2.0 g/L of citric acid, and more typically from about 0.4 g/L to about 1.6 g/L of citric acid.

The oral rehydration solutions of the present disclosure may comprise less than about 25 mEq/L of citrates, or less than about 20 mEq/L of citrates, or less than about 10 mEq/L of citrates, or less than about 8 mEq/L of citrates, or less than about 5 mEq/L of citrates. These amounts include citrates from any source, including citric acid; citric ester that can be hydrolyzed into citric acid or a citrate ion; or a citrate salt, such as potassium citrate, sodium citrate, and combinations thereof. It should be understood that the lower citrate amounts are typically found in higher pH solutions.

### Sodium Source

The oral rehydration solutions of the present disclosure further comprise sodium. The sodium in the oral rehydration solutions may be present as a cation of a salt. Examples of suitable sodium sources include sodium chloride, sodium phosphate, sodium citrate, sodium carbonate, sodium bicarbonate, sodium hydroxide, and combinations thereof.

The quantity of sodium ions typically used in oral rehydration solutions varies widely. For instance, typical oral rehydration solutions comprise from about 30 mEq/L to about 95 mEq/L of sodium. In contrast, the ORS of the present disclosure advantageously has a sodium content of from about 10 mEq/L to about 20 mEq/L, more preferably from about 12 mEq/L to about 18 mEq/L, more preferably about 14 mEq/L to about 16 mEq/L, and still more preferably about 15 mEq/L, which is an appropriate amount for administration to young infants.

### Zinc Source

In addition to dextrose and sodium, the oral rehydration solutions of the present disclosure further comprise a source of zinc. The presence of zinc in the ORS of the present disclosure helps support the immune system of children.

The source of zinc is generally not critical. Any zinc salt suitable for human consumption may be used in the oral rehydration solutions of this disclosure. Examples of suitable zinc sources include zinc gluconate, zinc sulfate, zinc chloride, zinc citrate, zinc bicarbonate, zinc carbonate, zinc hydroxide, zinc lactate, zinc acetate, zinc fluoride, zinc bromide, zinc sulfonate, and combinations thereof.

The amount of zinc used in the oral rehydration solutions of the present disclosure can vary widely. For example, the ORS of the present disclosure may comprise from about 1.8 mg to about 99 mg of zinc per liter of ORS, typically from about 1.8 mg/L to about 5 mg/L, from about 1.8 mg/L to about 3 mg/L, or from about 1.8 mg/L to about 2.2 mg/L.

### Water

The ORS of the present disclosure further comprises water. The amount of water present in the ORS will vary. Suitable amounts of water can readily be determined by one skilled in the art, and should be sufficient that, when combined with the other ORS components, will form an ORS having sodium, dextrose, and zinc in the amounts set forth herein.

### Optional Components

In addition to sodium, dextrose, and a zinc source, the oral rehydration solutions of this disclosure may contain all the necessary electrolytes and levels thereof required by the Food and Drug Administration for oral rehydration formulations sold in the United States. Further, the oral rehydration solutions may contain a source of carbohydrate in addition to dextrose, such as glucose or fructose. In some embodiments, the oral rehydration solutions of this disclosure may comprise water, dextrose, zinc ions, sodium ions, potassium ions, chloride ions, and citrate ions.

The oral rehydration solutions may contain a source of potassium ions. The potassium in an ORS may be present as an ion in the liquid, and may be in equilibrium with a salt. Examples of potassium salts include potassium chloride, potassium phosphate, potassium citrate, potassium carbonate, potassium bicarbonate, potassium hydroxide, and combinations thereof. The quantity of potassium present in the ORS can vary widely. However, as a general guideline, the ORS will typically contain from about 10 mEq/L to about 30 mEq/L of potassium, or from about 15 mEq/L to about 25 mEq/L of potassium.

The oral rehydration solutions will also typically contain a source of chloride. The chloride in an ORS may be present as an ion in the liquid, and may be in equilibrium with a salt. Examples of suitable chloride salts include, but are not limited to sodium chloride, potassium chloride, calcium chloride, magnesium chloride, and combinations thereof. The amount of chloride present in the ORS may vary. Typically, the ORS will comprise chloride in an amount of from about 30 mEq/L to about 80 mEq/L, but may comprise chloride in an amount as low as about 15 mEq/L.

The oral rehydration solutions may also optionally include a source of carbohydrate other than dextrose. Any carbohydrate suitable for use in oral rehydration solutions may be used in the oral rehydration solutions of the present disclosure. The carbohydrates may be simple and/or complex carbohydrates, including monosaccharides, disaccharides, oligosaccharides, and polysaccharides. Specific examples of suitable carbohydrates include, but are not limited to, glucose, fructooligosaccharides, galacto-oligosaccharides, fructose and glucose polymers, corn syrup, high fructose corn syrup, sucrose, maltodextrin, lactose, maltose, amylose, glycogen, galactose, allose, altrose, mannose, gulose, idose, talose, ribose, arabinose, lyxose, ribose, xylose, erythrose, threose, and combinations thereof. Preferably, the carbohydrates are either glucose alone or glucose combined with maltodextrin.

The levels of carbohydrates present in an ORS intended for the treatment of children with acute diarrhea are typically between about 11 g/L to about 25 g/L (about 1% to about 2.5% by weight). These levels are sufficient to permit maximum glucose-coupled sodium absorption. In cases of acute diarrhea, excessive amounts of carbohydrate are typically not desirable, as non-absorbed carbohydrates may exacerbate the fluid and electrolyte losses, producing osmotic diarrhea. In contrast, in the oral rehydration solutions of the present disclosure, which may be administered to children with fever but not diarrhea, the levels of carbohydrates are higher in order to provide adequate calories to prevent ketosis. For instance, the carbohydrate levels in the oral rehydration solutions of the present disclosure may be up to about 6% by weight.

An oral rehydration solution of the present disclosure may include one or more additional ingredients. Examples of additional ingredients in an ORS or ORM include flavorants, colorants, preservatives, excipients, gelling agents, indigestible oligosaccharides, amino acids, calcium, vitamins, dietary supplements, and combinations thereof. Preferably the amount of any additional ingredients in an ORS or ORM is such that the primary ingredients remain within the desired ranges.

A flavorant may be present to add or modify a flavor in the oral rehydration solution, or to enhance its palatability, especially in a pediatric population. Examples of suitable flavorants include anise oil, cinnamon oil, vanilla, vanillin, cocoa, chocolate, menthol, grape, fruit punch flavoring, bubble gum flavoring, peppermint oil, oil of wintergreen, clove oil, bay oil, anise oil, eucalyptus, thyme oil, cedar leaf oil, oil of nutmeg, oil of sage, oil of bitter almonds, cassia oil, citrus oils such as lemon, orange, lime and grapefruit oils, and fruit essences, including apple, pear, peach, berry, wildberry, date, blueberry, kiwi, strawberry, raspberry, cherry, plum, pineapple, and apricot.

Artificial sweeteners may also be added to complement the flavor. The concentration of sweetener in the ORS may be from 0.01 to 0.5 grams per Liter (g/L). Useful artificial sweeteners include saccharin, nutrasweet, sucralose, aspartame, acesulfane-K (ace-K), and the like. Preferably the sweetener is chlorinated sucrose. Chlorinated sucrose is a no-calorie sweetener made by replacing three of the hydroxy groups (OH) of the sugar molecule with chlorine (Cl). The chlorine atoms are tightly bound to the sugar molecule, thus making it exceptionally stable. This stability is believed to prevent the body from digesting the molecule, allowing the chlorinated sugar molecules to pass through the body unchanged. The chlorination process may create multiple isomers of the sugar, depending on the reaction conditions and other variables. Sucralose is the common name for one of the isomers resulting from the chlorination process. Sucralose is considered to be about 600 times sweeter than sugar and to have a medium intensity of sweetness coupled with a relatively long-lasting sweetness in the mouth.

The presence of a sweetener in the formulation may allow for a decrease in the amount of glucose or similar carbohydrate in the ORS. Preferably a sweetened ORS contains a decreased level of glucose or similar carbohydrate, for example, from about 1.2 to about 1.8 wt % (67 to 100 mEq/L for glucose). An ORS having a decreased amount of glucose or similar carbohydrate may provide nutritional benefits, as well as improved patient or consumer acceptance. A decrease in glucose content can provide an ORS having fewer calories. Edible products marketed for children, including pediatric ORS's, typically include fructose instead of or in addition to glucose, as this can provide an increased level of sweetness that is preferred by children.

A colorant may be present to add or modify a color in the oral rehydration solutions. Examples of colorants include FD&C Red No. 3, FD&C Red No. 20, FD&C Yellow No. 6, FD&C Blue No. 2, D&C Green No. 5, FD&C Orange No. 5, D&C Red No. 8, caramel, ferric oxide, pigments, dyes, tints, titanium dioxide, grape skin extract, beet red powder, beta carotene, annato, carmine, turmeric, paprika, and the like.

A preservative may be present to provide a longer shelf life to a pre-packaged ORS, or to extend the potability lifetime of an ORS. Examples of suitable preservatives include, but are not limited to potassium sorbate and sodium benzoate.

A gelling agent may be present in the ORS, such that the ORS can be formed into a gel, such as a flowable gel or a self-supporting gel. ORS gels may provide improved patient compliance in consuming an ORS, especially in a pediatric population. Gelled rehydration solutions are described in U.S. Pat. No. 6,572,898, herein incorporated by reference. Gelling agents may be included in the ORS in amounts of from about 0.05 to about 50% (w/w).

An indigestible oligosaccharide may optionally be included in the ORS. Indigestible oligosaccharides may provide a benefit to the gastrointestinal tract. For instance, indigestible oligosaccharides may help to suppress the growth of pathogenic organisms such as Clostridium difficile, and/or to selectively promote the growth of a nonpathogenic microbial flora. Examples of suitable indigestible oligosaccharides include fructo-ologosaccharides (FOS), galacto-oligosaccharides (GOS), inulins such as raftilose, and xylooligosaccharides. In cases of patients with fever and no diarrhea, an indigestible oligosaccharide like FOS or GOS may support the GI health and may provide immune benefits. An indigestible oligosaccharide may be present in the ORS in an amount of from about 1 g/L to about 8 g/L.

Calcium or a calcium containing substance may also be included in an ORS of the present disclosure. Examples of suitable calcium containing substances include calcium chloride, calcium oxide, calcium hydroxide, calcium carbonate, calcium orthophosphate (including mono-, di- and tricalcium phosphate), calcium lactate, calcium gluconate, calcium citrate, calcium acetate, calcium ascorbate, calcium tartarate, calcium malate and mixtures of these. The calcium may be included in the ORS in amounts of from about 5 mEq/L to about 30 mEq/L, from about 10 mEq/L to about 25 mEq/L, or from about 15 mEq/L to about 20 mEq/L.

### Methods of Manufacture

The ORS of the present disclosure can be manufactured using techniques well known to those skilled in the art. For instance, the ORS may be prepared by combining the non-aqueous (i.e. “dry”) ingredients of the ORS, for example by dry blending, and dispersing the dry ingredients in a suitable amount of water to provide a liquid having the appropriate concentrations of ingredients, as set forth herein. Alternately, one or more of the dry ingredients may be added separately to the water. The ORS may optionally be heated to the appropriate temperature to dissolve all the ingredients, packaged, and sterilized to food grade standards as is known in the art.

The oral rehydration solutions are generally heat sterilized either by a retort process, an aseptic process, or a hot fill process. In one aspect, the method of preparing the ORS further comprises adjusting the pH of the ORS by adjusting the amount of dextrose in the ORS prior to sterilization.

A typical retort process involves introducing the ORS into a metal or plastic container, sealing the container, and then heating the sealed container for a time period and to a temperature sufficient for sterilization. Aseptic sterilization involves separately sterilizing a metal or plastic container and the ORS and then combining the sterilized container and ORS in a clean room environment and sealing the container. In a hot fill process, the container is tilled with the ORS and sealed at product temperatures above room temperature.

More specifically, in the retort sterilization method, the ORS is usually preheated and then filled into a clean can, hermetically sealed, and placed in a steam chamber and sterilized, normally at about 121° C. for about 15 to about 45 minutes. The batch is then cooled and the retort filled with a new batch. Because sterilization takes place after filling, the need for aseptic handling is eliminated, although heat resistant plastic (or another heat resistant material) must be used due to the high temperatures involved. In one specific retort sterilization embodiment, a hydrostatic tower method is utilized and includes conveying slowly the sealed containers through successive heating and cooling zones in a sterilizer. The zones are dimensioned to correspond to the required temperatures and holding times in the various treatment stages.

In the aseptic sterilization method, the ORS is sterilized and a container is separately sterilized. The ORS may be sterilized utilizing a heating process, for example. The container may be sterilized by spraying the interior wall of the container with hydrogen peroxide and then drying the interior wall. Once the container and the ORS have both been sterilized, the ORS is introduced into the container in a clean room environment and the container sealed.

Hot fill processes alone can be used to sterilize a high acid product (approximately below pH 4.6). In hot fill sterilization, the container is filled with ORS and the container is sealed at approximately 180° F. The filled container is then rotated end-over-end so that the hot ORS contacts all surfaces and, finally, it is held hot for approximately five to ten minutes to kill all viable microorganisms. Microorganisms which are viable at low pH are molds and yeasts. If the product is a low acid product, approximately above pH 4.6, the hot fill process does not produce adequate sterility. Terminal sterilization is used to kill harmful organisms potentially viable above pH 4.6. Terminal sterilization kills potentially viable organisms by raising product and container temperatures to the equivalent of 250° F. for times equivalent to at least 3 minutes, more often, in excess of 10 minutes as determined using established practices to calculate sterilization process time as a function of product temperature history. The time the product and container are held at an elevated temperature can be reduced markedly by using sterilizer and product temperatures in excess of 250° F. Sterilizer and product temperatures well in excess of 250° F. are commonly used in order to reduce sterilization process time.

### Product Form

An ORS may be packaged in a container such as a glass or plastic bottle, a plastic pouch, or a paper-based carton. In one example, an ORS may be formed by combining water with the remaining ORS ingredients, agitating and/or heating the mixture to dissolve the ingredients, and then packaging the ORS in a container. The ORS may be sterilized before or after being packaged, such as by retort, aseptic, or hot fill sterilization, as discussed above. The ORS may be packaged in a container that includes an oxygen barrier, an oxygen scavenger, and/or an ultraviolet radiation barrier. A single package of ORS may contain a single serving, such as 12 fl.oz. (0.35 L) or 1 L. A single package of ORS may contain multiple servings, such as multiples of 12 fl.oz. (0.35 L) or of 1 L.

An ORS may also be packaged in non-liquid forms, provided the ORS has undergone heat sterilization. In one example, an ORS may be packaged as a gel containing one or more gelling agents as described above. In another example, an ORS may be packaged as a frozen solution. Frozen ORS may be in the form of ice cubes, ice on a stick (i.e. “freezer pop”), crushed ice, or shaved ice, for example. Advantageously, frozen ORS may provide improved patient compliance in consuming an ORS, particularly in pediatric populations. Frozen ORS is disclosed, for example, in U.S. Pat. No. 5,869,459, herein incorporated by reference.

### Methods of Use

The oral rehydration solutions of the present disclosure may be used to prevent dehydration in an individual, particularly in individuals suffering from fever. Thus, in one aspect, the present disclosure is directed to a method for preventing dehydration from fever. The method comprises orally administering an ORS of the present disclosure to an individual at risk of developing dehydration, or more particularly, an individual at risk of developing dehydration from fever or other illnesses, not including diarrhea or vomiting. The individual may be, for example, an infant, child, or adult, but preferably is a child. The method may further comprise preparing an ORS of the present disclosure using any of the methods described herein.

The amount of ORS administered to the individual will vary. Typically, from about 200 mL to about 4000 mL of the ORS may be administered every 4 to 6 hours, depending on the individual's weight and/or age. Exemplary doses of ORS that may be administered every 4 to 6 hours include: from about 200 mL to about 400 mL for individuals weighing less than about 5.5 kg or who are up to about 6 months old; from about 400 mL to about 600 mL for individuals weighing from about 5.5 kg to about 9.5 kg or who are about 6 to about 12 months old; from about 600 mL to about 800 mL for individuals weighing from about 9.5 kg to about 13 kg or who are about 12 months to about 3 years old; from about 800 mL to about 1000 mL for individuals weighing from about 13 kg to about 20 kg or who are about 3 years to about 8 years old; from about 1000 mL to about 2000 mL for individuals weighing from about 20 kg to about 40 kg or who are about 8 years old to adult; or from about 2000 mL to about 4000 mL for individuals weighing over about 40 kg or who are adults.

ORS may be administered in a variety of different forms, depending upon patient preference. For example, some children will consume ORS more readily if it is frozen, like a freezer pop. The ORS of this solution may be administered as frozen ORS if the patient desires such a choice. Other examples of suitable product forms are set forth herein, such as liquid and gels.

### EXAMPLES

The following examples are provided to illustrate one or more specific embodiments of the disclosure. The examples are given solely for the purpose of illustration and are not to be construed as limitations of the present disclosure, as many variations thereof are possible without departing from the spirit and scope of the disclosure.

### Examples 1-6

The following examples illustrate oral rehydration solutions comprising 15 mEq/L of sodium and varying amounts of dextrose. The ingredients listed in Table I were combined to form a stock solution having 15 mEq/L of sodium, but no dextrose. The exemplary oral rehydration solutions were prepared by adding dextrose, citric acid anhydrous, and/or water to the stock solution in the amounts set forth in Tables 2A and 2B.

TABLE 1
15 mEq/L Stock Solution

| Ingredient | Quantity |
|---|---|
| Water (lb) | 65.5 |
| Potassium citrate (g) | 31.6 |
| Sodium chloride (g) | 25.5 |
| Zinc gluconate (g) | 0.433 |
| Total weight (lb) | 65.63 |

TABLE 2A
Oral rehydration solutions

| | Example | Example | Example | Example | Example | Example |
|---|---|---|---|---|---|---|
| Ingredient | 1 | 2 | 3 | 4 | 5 | 6 |
| 15 mEq stock (lb) | 9.3 | 9.3 | 9.3 | 9.3 | 9.3 | 9.3 |
| Dextrose | 292.6 | 234.1 | 175.5 | 117 | 78 | 53.5 |
| monohydrate (g) | | | | | | |
| Citric acid anhydrous | 3.9 | 3.9 | 3.9 | 3.9 | 3.9 | 3.9 |
| (g) | | | | | | |
| Water (g) | — | 60 | 136 | 195 | 235 | 258 |
| Total weight (lb) | 10 | 10 | 10 | 10 | 10 | 10 |
| Total Na (mEq/L) | 15.0 | 15.0 | 15.0 | 15.0 | 15.0 | 15.0 |
| Total K (mEq/L) | 9.6 | 9.6 | 9.6 | 9.6 | 9.6 | 9.6 |
| Total Cl (mEq/L) | 15.0 | 15.0 | 15.0 | 15.0 | 15.0 | 15.0 |
| Total citrate (mEq/L) | 23.5 | 23.5 | 23.5 | 23.5 | 23.5 | 23.5 |
| Total dextrose (g/L) | 60.0 | 48.0 | 36.0 | 24.0 | 16.0 | 11.0 |

TABLE 2B
Oral rehydration solutions

| | Example | Example | Example | Example | Example | Example |
|---|---|---|---|---|---|---|
| Ingredient | 7 | 8 | 9 | 10 | 11 | 12 |
| 15 mEq stock (lb) | 9.3 | 9.3 | 9.3 | 9.3 | 9.3 | 9.3 |
| Dextrose | 292.6 | 234.1 | 175.5 | 117 | 78 | 53.5 |
| monohydrate (g) | | | | | | |
| Citric acid anhydrous | 3.74 | 3.71 | 3.73 | 3.67 | 3.69 | 3.66 |
| (g) | | | | | | |
| Water (g) | — | 60 | 136 | 195 | 235 | 258 |
| Total weight (lb) | 10 | 10 | 10 | 10 | 10 | 10 |
| Total Na (mEq/L) | 15.0 | 15.0 | 15.0 | 15.0 | 15.0 | 15.0 |
| Total K (mEq/L) | 9.6 | 9.6 | 9.6 | 9.6 | 9.6 | 9.6 |
| Total Cl (mEq/L) | 15.0 | 15.0 | 15.0 | 15.0 | 15.0 | 15.0 |
| Total citrate (mEq/L) | 23.0 | 22.9 | 22.9 | 22.7 | 22.8 | 22.7 |
| Total dextrose (g/L) | 60.0 | 48.0 | 36.0 | 24.0 | 16.0 | 11.0 |

### Example 7

In this example, the pre- and post-retort pH of the oral rehydration solutions of Examples 1-6 was determined and compared to the pre- and post-retort pH of control oral rehydration solutions. The control oral rehydration solutions were prepared by combining the ingredients in the amounts as set forth in Table 3.

TABLE 3
Control oral rehydration solutions

| | Control | Control | Control | Control | Control | Control | Control |
|---|---|---|---|---|---|---|---|
| Ingredient | 1 | 2 | 3 | 4 | 5 | 6 | 7 |
| Water (lb) | 9.3 | 9.3 | 9.3 | 9.0 | 9.1 | 9.2 | 9.9 |
| Sodium chloride | 8.2 | 9.3 | 7.9 | 3.9 | 3.9 | 3.9 | 3.9 |
| (g) | | | | | | | |
| Potassium citrate | 4.6 | 10.2 | 6.8* | 4.6 | 4.6 | 4.6 | 4.6 |
| (g) | | | | | | | |
| Sodium citrate, | 0.2 | 5.1 | 13.0 | 0 | 0 | 0 | 0 |
| dihydrate (g) | | | | | | | |
| Dextrose | 292.6 | 292.6 | 292.6 | 439.1 | 390.1 | 341.6 | 24.5 |
| monohydrate (g) | | | | | | | |
| Citric acid | 12.2 | 12.2 | 12.2 | 3.9 | 3.9 | 3.9 | 4.0 |
| anhydrous (g) | | | | | | | |
| Zinc gluconate | 0.066 | 0.066 | 0.066 | 0.066 | 0.066 | 0.066 | 0.066 |
| (g) | | | | | | | |
| Total weight (lb) | 10 | 10 | 10 | 10 | 10 | 10 | 10 |
| Total sodium | 30 | 45 | 60 | 15 | 15 | 15 | 15 |
| (mEq/L) | | | | | | | |
| Total dextrose | 60 | 60 | 60 | 90 | 80 | 70 | 5 |
| (g/L) | | | | | | | |

*potassium chloride was used instead of potassium citrate

The pH of the oral rehydration solutions from Examples 1-6 and Controls 1-7 was determined before and after subjecting the oral rehydration solutions to retort processing. The results are set forth in Table 4.

TABLE 4
Pre- and post-retort pH

| | | | | | Difference |
|---|---|---|---|---|---|
| Oral | | | Pre- | Post- | between pre- |
| rehydration | Sodium | Dextrose | retort | retort | and post- |
| solution | (mEq/L) | (g/L) | pH | pH | retort pH (Δ) |
| Example 1 | 15 | 60 | 4.16 | 4.03 | 0.13 |
| Example 2 | 15 | 48 | 4.11 | 4.00 | 0.11 |
| Example 3 | 15 | 36 | 4.20 | 4.02 | 0.18 |
| Example 4 | 15 | 24 | 4.17 | 3.94 | 0.23 |
| Example 5 | 15 | 16 | 4.18 | 3.97 | 0.21 |
| Example 6 | 15 | 11 | 4.17 | 3.94 | 0.23 |
| Control 1 | 30 | 60 | 4.08 | 3.86 | 0.22 |
| Control 2 | 45 | 60 | 4.18 | 4.00 | 0.18 |
| Control 3 | 60 | 60 | 4.16 | 3.98 | 0.18 |
| Control 4 | 15 | 90 | 4.14 | 3.98 | 0.16 |
| Control 5 | 15 | 80 | 4.12 | 4.00 | 0.12 |
| Control 6 | 15 | 70 | 4.18 | 3.99 | 0.19 |
| Control 7 | 15 | 5 | 4.11 | 3.94 | 0.17 |

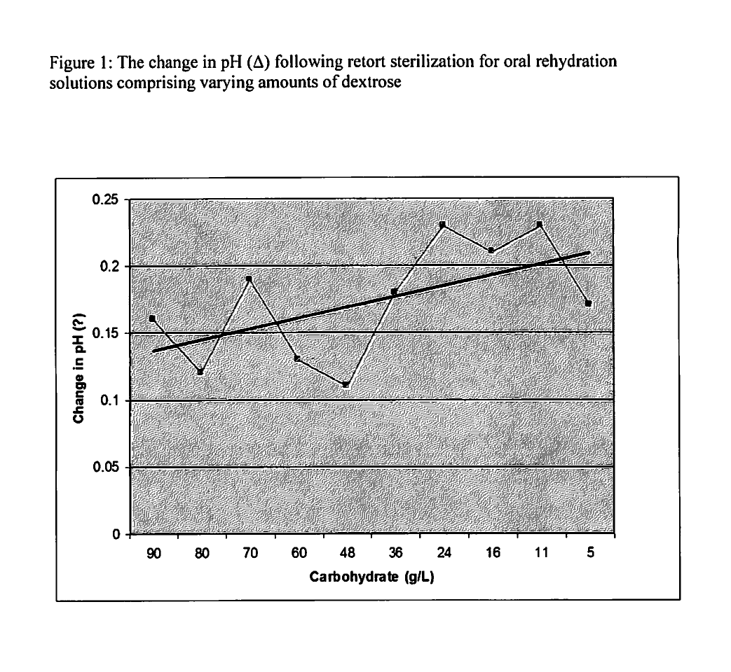

As can be seen from Table 3, all oral rehydration solutions showed a decrease in pH following retort. Linear regression analysis was applied to the difference between pre- and post-retort pH (Δ) for the oral rehydration solutions comprising 15 mEq/L sodium, and the results are shown in FIG. 1. As can be seen from FIG. 1, there is a general trend towards a greater difference in pre- and post-retort pH (Δ) (i.e., a greater drop in pH following retort processing) as the amount of dextrose in the ORS decreases, for oral rehydration solutions comprising 15 mEq/L of sodium. The difference in pre- and post-retort pH for ORS comprising 15 mEq/L and 11-36 g/L of dextrose was comparable to or larger than that observed for the control oral rehydration solutions comprising 30-60 mEq/L of sodium.

### CONCLUSION

The data set forth herein show that in oral rehydration solutions comprising 15 mEq/L of sodium, the pH of the ORS following retort can be controlled by adjusting the amount of dextrose present in the ORS. Surprisingly, it has been discovered that higher dextrose levels do not result in a greater drop in pH following retort when compared to lower dextrose levels in 15 mEq/L sodium ORS. The data also indicates that oral rehydration solutions comprising 15 mEq/L sodium and 11-36 g/L of dextrose exhibited a difference between pre- and post-retort pH that was comparable to or larger than that observed for oral rehydration solutions comprising higher amounts of sodium (30-60 mEq/L).

What is claimed is:

1. An oral rehydration solution comprising about 12 mEq/L to about 18 mEq/L of sodium; from about 5 g/L to about 90 g/L of dextrose; a zinc source; and less than about 25 mEq/L of citrates, wherein the oral rehydration solution has been heat sterilized.

2. The oral rehydration solution of claim 1, wherein the oral rehydration solution comprises from about 11 g/L to about 60 g/L of dextrose.

3. The oral rehydration solution of claim 1, wherein the oral rehydration solution comprises from about 11 g/L to about 36 g/L of dextrose.

4. The oral rehydration solution of claim 1, wherein the oral rehydration solution comprises from about 1.8 mg/L to about 99 mg/L of zinc.

5. The oral rehydration solution of claim 1, wherein the oral rehydration solution has been sterilized using retort sterilization, aseptic sterilization, or hot fill sterilization.

6. The oral rehydration solution of claim 5, wherein the oral rehydration solution has a pH of from about 3.0 to about 5.5.

7. The oral rehydration solution of claim 5 wherein the difference between the pH of the oral rehydration solution prior to sterilization and the pH of the oral rehydration solution following sterilization is from about 0.18 to about 0.23.

8. The oral rehydration solution of claim 1, wherein the oral rehydration solution comprises from about 0.3 g/L to about 2.0 g/L of citric acid.

9. The oral rehydration solution of claim 1 further comprising from about 10 mEq/L to about 30 mEq/L of potassium and from about 30 mEq/L to about 80 mEq/L of chloride.

10. The oral rehydration solution of claim 1 further comprising at least one ingredient selected from the group consisting of flavorants, colorants, preservatives, excipients, gelling agents, indigestible oligosaccharides, amino acids, calcium, vitamins, dietary supplements, and combinations thereof.

11. A method of making an oral rehydration solution comprising:
combining suitable amounts of water, dextrose, a sodium source, and a zinc source to form an oral rehydration solution comprising about 12 mEq/L to about 18 mEq/L of sodium, from about 11 g/L to about 60 g/L of dextrose, the zinc source, and less than about 25 mEq/L of citrates; and heat sterilizing the oral rehydration solution.

12. The method of claim 11 further comprising adjusting the pH of the sterilized oral rehydration solution by adjusting the amount of dextrose in the oral rehydration solution prior to sterilization.

13. A method of preventing dehydration comprising:
preparing an oral rehydration solution comprising about 12 mEq/L to about 18 mEq/L of sodium, from about 11 g/L to about 60 g/L of dextrose, a zinc source, and less than about 25 mEq/L of citrates, wherein the oral rehydration solution has been heat sterilized; and orally administering the sterilized oral rehydration solution to an individual at risk of developing dehydration.

14. The method of claim 13 wherein the individual is a child.

15. The method of claim 13 wherein the sterilized oral rehydration solution comprises from about 11 g/L to about 36 g/L of dextrose.

16. The method of claim 13 wherein the individual is suffering from fever.

17. The method of claim 16 wherein the oral rehydration solution comprises about 15 mEq/L of sodium.

18. An oral rehydration solution comprising about 15 mEq/L of sodium; from about 5 g/L to about 90 g/L of dextrose; a zinc source; and less than about 25 mEq/L of citrates, wherein the oral rehydration solution has been heat sterilized.

19. The oral rehydration solution of claim 18, wherein the oral rehydration solution comprises from about 11 g/L to about 60 g/L of dextrose.

20. The oral rehydration solution of claim 18, wherein the oral rehydration solution has been sterilized using retort sterilization, aseptic sterilization, or hot fill sterilization.

21. The oral rehydration solution of claim 18, wherein the oral rehydration solution has a pH of from about 3.0 to about 5.5.
